(19) United States
(12) Patent Application Publication
(10) Pub. No.: US 2006/0173712 A1
(43) Pub. Date: Aug. 3, 2006

(54) Portable medical information system

(76) Inventor: Dirk Joubert, Alpharetta, GA (US)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 11/270,978

(22) Filed: Nov. 12, 2005

Related U.S. Application Data
(60) Provisional application No. 60/627,368, filed on Nov. 12, 2004.

(51) Int. Cl. G06Q 10/00; A61B 5/00

(52) U.S. Cl. 705/2; 600/300

(57) ABSTRACT

A computerized system for monitoring and maintaining the health of a person comprising the steps of (a) obtaining parameter data from the patient and inputting the parameter data into a computer database; (b) analyzing the parameter data using a computerized statistical modeling technique module and a computerized adaptive expert system shell for the prediction of a health event in the lifetime of the patient; (c) using the analyzed data to developed a health maintenance schedule for the patient, and (d) embedding and/or linking such data onto a microprocessor powered smart card.

### STATEMENT OF RELATED APPLICATIONS

This patent application is based on and claims priority on U.S. provisional patent application No. 60/627,368 having a filing date of 12 Nov. 2004.

### BACKGROUND OF THE INVENTION

1. Technical Field

The present invention generally relates to the field of manipulating electronic medical information and more specifically relates to the field of storing, retrieving, using and updating medical information using a portable smart card that can be carried by an individual and accessed at the point of providing medical services. The present invention further generally relates to the field of medical diagnoses, prognoses, prevention, and health maintenance and more specifically relates to the field of storing, retrieving, using and updating recent medical information such as hospital or physician visits, diagnoses, evaluations and prescriptions initiated or spawned by a portable smart card that can be carried by an individual and accessed at the point of providing medical services so as to provide a current medical practitioner with up-to-date information about the patient.

2. Prior Art

Medical information is stored in individual records, generally paper, at individual physicians' offices, in hospitals, and in insurance company databases. For the most part, the storage of medical information is widely dispersed, even for a single patient. Each medical site that a patient has visited generally has a file on the patient. Although the patient's insurance company may have a more complete record file for a patient, there is no guarantee that such a file is complete or even up to date, especially because health insurance is portable and patient's often switch insurance companies, sometimes as often as every year.

When a patient visits a new physician, the physician takes a medical history from the patient. Generally, this medical history is what the patient can remember. In some instances, the new physician is able to obtain the patient's past records and medical charts from previous physicians. However, this process may take days or weeks to complete and the new physician then needs to incorporate the old records into a new record. Often this consists solely of placing the old records in the new paper file.

In many instances, when a patient is seen by a new or different physician or at a new or different hospital, the new or different physician or hospital does not have the benefit of knowing the patient's medical history. If the patient has a good memory or is conscious, the patient can provide details about his or her medical history to the new physician or hospital. However, if the patient has a poor memory, is in shock, or is unconscious, the new physician or hospital may need to start treating the patient without the benefit of a medical history. This can lead to unnecessary, duplicative treatment, and/or incorrect treatment or diagnoses.

Thus, it can be see that there is a need for a new method and system of maintaining patient records in a portable device that the patient can keep with them such that when the patient visits or is taken to a new or different physician or hospital, the new or different physician or hospital will have the benefit of retrieving and/or viewing the patient's medical history. There also is a need for a new method and system of maintaining a patient's basic information, such as names, contacts, allergies, chronic medication, contraindications, insurance companies, and the like in a portable device that the patient can keep with them and present to a physician, hospital or any medical service providing facility to facilitate diagnosis and treatment. The present invention is directed to these and other needs.

### BRIEF SUMMARY OF THE INVENTION

The present invention was developed in part to address recommendations defined by the US Federal Government's Healthcare Reform Initiative adopted by the US House of Representatives in June 2003. The objectives of the US Department of Health and Human Services (HHS) target improvement of clinical decisions, reduction of duplicative data entry, reduction of medical errors or Rx, and easy and rapid access to patient medical information.

The present invention provides definitive verification of patient, physician and patient benefits and includes:

1. A personal identification, biometric authentication and additional personal data sections, secured via biometric verification without the need for a mainframe or database of any kind;

2. A medical benefit, risk and management data section, accessible only by healthcare professionals including files for referrals, medical certificates and messages to facilitate better communication between healthcare professionals; and

3. A payment portion that enables e-purse and credit and debit payments.

Another valuable feature of the system is its multiple purpose use. Cards that are issued for medical purposes could concurrently be used for Worker's Compensation programs, secured access control, time and attendance, stored value of Health Savings Account credits (HSA), retail loyalty points, etcetera.

An embodiment of the present invention comprises the use of an adaptive expert system (such as the Solvatio® system) to provide users with an auditable and validatable trail of service provision through an embedded graphic “derivation map” on a smart card that shows symptom-to-diagnosis-to treatment. Additional benefits include information accessibility in medical emergencies, rapid electronic claims validation, processing and payment. The system also features tracking of under and over service provision, accrued totals for services by specialty, reduces fraud, and facilitates substantial timesaving in the administrative process both at practice and payer level.

The process of the present invention comprises the following features:

1. HIPAA (US Health Insurance Portability and Accountability Act of 1996) required information of participants is collected and stored on a smart card;

2. Hospital admission is facilitated by simply waving the patient's smart card near a reader;

3. In-facility procedures are tracked and recorded at time of provision, all with positive verification of patient ID;

4. The patient authorizes release/access to their personal information through a fingerprint or voice biometric (a fingerprint biometric satisfies the HIPAA requirements);

5. The patient's case is electronically retrieved from their case file in the expert system and routed to the attending physician's workstation. This updateable case/progress note provides the platform that guides the clinical encounter on presentation of the smart card; and

6. Upon discharge all information pertinent to the clinical encounter is automatically written to the patient's card and updated in the expert system.

The present invention has a pricing model that is based on pay-per-use, negating the need for large capital expenditures by the user. The present invention can generate revenue on the sale or leasing of the smart cards, readers, support and maintenance, as well as on-going transaction fees.

### DETAILED DESCRIPTION OF THE PREFERRED EMBODIMENTS

1. Overview.

The present invention comprises a smart card for identification and biographic information, medical management, and e-purse applications, and is based on extensive development to provide portability of personal Electronic Medical Information (EMI) without the need for a centralized database. The system was developed to:
Encompass, interact and interfaces on primary, secondary and tertiary healthcare levels.
Allow effective communication between all role-players.
Identify and authenticate a patient and his/her healthcare benefits at point of service.
Secure effective data capturing of the clinical encounter, based on standardized international codes for diagnosis, treatments, medicines etcetera via point of service medical, clinical and administrative management systems linked to databases.
Secure effective database storage, management and mining.
Provide clinical guidelines, protocols and patient-doctor performance based statistics, at point of service, based on clinical guidelines and protocols, and the latest international standards and outcome reviews.
Apply the best clinical and financial risk management tools and standards available.
Facilitate fast effective payment in a variety of models, for services rendered at service provider level, based on various benefit options, protocol compliance and performance of patient and service provider.
Facilitate quality, secure, sustainable, cost effective and well-managed healthcare delivery to all citizens.

Electronic access of patient information facilitates updating of records simply through the presentation of the card to a reader and, has functionality that features interfacing to online eligibility and service provision parameters to compare and validate the patient's status embedded on the card. At clinical encounter level the card can be configured to act as the initiator of a clinical encounter ensuring that the attending physician/pharmacist is informed as to contraindications, chronic medication, and allergies. New clinical encounter data—the case—is written to the card utilizing standard international codes to indicate the date, patient-number, service provider-number, codes for diagnosis (CPT4, ICD9/10), clinical services, procedures, medication (e-scripting) and cost. This enables the user to carry his/her critical and important identification, clinical and other data on a single contactless smart card for use within the medical community without the need for duplicative data capture/re-entry. Access to such data is protected through the use of various security features, such as fingerprint verification, and the need to go online or access a database is in most cases eliminated. All available options, such as managing fee for service, managed fee for service, group and individual capitation, short term Insurance, hospital plans and combinations of the above are customizable.

The present invention can be interfaced to a custom practice management system as part of the invention, or to established and leading primary and secondary healthcare provider practice management software systems, through a dynamic free issue software middleware link (SmartLink), clinic, hospital, administrator, and pharmacy back-end information technology (IT) systems, providing users with a fully integrated and seamless end to end integrated mobile healthcare solution. In addition:
The present invention can be customized to link to a variety of switches/bridges for electronic claims processing.
On the pharmacy level the present invention can be interfaced to leading pharmacies and Pharmacy Benefit Management Companies (PBM) software systems.
Electronic Funds Transfer (EFT) can be managed through a traderoot switch.
The microprocessor-based smart card features an on-board operating system to easily incorporate new applications onto existing smart cards, as well as revising existing applications in a secure manner via rapidly executable software routines.
The ability to alter the functionality of the smart card through remote software upgrades enhances the flexibility of the smart card as an e-commerce or mobile computing platform and data repository.
Up to 16 different virtual cards with 256 individual files can be run on any single smart card, providing for up to 16 different applications; a feature that facilitates the deployment of other value-added applications on the same physical platform making the system multi-use. As an example, a smart card issued for medical applications may be used for time and attendance, secured access, mass transit, driver's license, etcetera.
Real time Connectivity to the present invention is facilitated either through standard Internet/intranet or, via the Panamsat 7 Ku Band satellite backbone.
   2. General System Features and Operation.

Generally speaking the invention comprises a smart card containing a memory chip, a reader for reading the information stored on the smart card, a writer for writing (storing) information on the smart card, and a computer for storing individual and/or combined records regarding individuals and/or groups of individuals. The reader and the writer can be a combined reader/writer device. More specifically, the smart card stores information regarding the individual to whom the smart card is assigned and, in the preferred embodiment, personal and medical information about the individual.

In a preferred embodiment, the smart card comprises a memory chip with memory sufficient to store personal data regarding the individual and enough medical data to allow a medical practitioner to glean sufficient knowledge about the individual to make a more informed diagnosis. For example, the personal data can include, but is not limited to, the person's name, address, age, recent vital statistics, allergies, and insurance information. The medical data can include, but is not limited to, the results of the individual's most recent visits to medical practitioners, any recent surgeries or other medical treatments, and any other critical medical information about the individual that a medical practitioner would need to know before diagnosing and treating the individual. For example, the smart card can include, and/or retrieve the results, diagnoses, procedures, and treatments of the individual's last sixteen visits to a medical practitioner.

In a preferred embodiment, the smart card reader comprises a contactless reading device such that the reader can read the information on the smart card merely by placing the smart card proximal to the reader. The reader can comprise or can be attached to a device that allows the display, retrieval, and/or manipulation of the information on the smart card.

In a preferred embodiment, the smart card writer comprises a contactless writing device such that the writer can write information to the smart card merely by placing the smart card proximal to the writer. The writer and the reader can be a combined reader/writer device.

In a preferred embodiment, the system comprises a biometric verification device. For example, the biometric verification device can be a fingerprint reader, a voice recognition device, or a retinal scan device. The verification device allows the system to read the information on the smart card and provides a failsafe against reading the information on the smart card without the presence and permission of the individual.

In a preferred embodiment, the system comprises a remote computer capable of storing and/or manipulating the information regarding the individuals, such as a computer running database manipulation software. The remote computer can display the information received from the smart card for the medical practitioner. Similarly, the remote computer can receive input from the medical practitioner regarding the current visit and any diagnoses and treatments made by the medical practitioner. The remote computer than can send this updated information to the writer for writing the updated information back onto the smart card.

In a preferred embodiment, the system can comprise a centralized computer capable of storing and/or manipulating information regarding the individual or groups of individuals, such as a computer running database manipulation software. Preferably, this centralized computer is able to communicate with the remote computer either through the Internet, via wired or wireless networking, and/or through an intranet. The centralized computer is useful for when the individual cannot or is not visiting the individual's regular medical practitioner in that the centralized computer can transmit the individual's information to the remote computer where the individual now is located, or can allow the remote computer at the individual's regular medical practitioner to interface with the remote computer where the individual is now located.

In a preferred embodiment, both the remote computer and/or the centralized computer store the individuals' information in separate discrete files. Using separate discrete files for each individual has many advantages including greater privacy, smaller files more easily transmitted over the Internet/intranet, and less data loss if a file becomes corrupted. Alternatively, the remote computer and/or the centralized computer can maintain all the individuals' records in a single or multiple large combined databases. The remote computer and the centralized computer can be a single centralized computing device that the reader/writer communicates with directly over the Internet/intranet.

In a preferred embodiment, the computer (either or both the remote computer and the centralized computer) runs expert analysis software. The expert analysis software manipulates the individual's information and assists the medical practitioner in making a diagnosis and prescribing treatment. Generally, the expert analysis software manipulates the individual's information and though a set of rules, or by comparison to similar cases, arrives at a diagnosis and treatment regimen based on the medical history of the individual stored on the computer. Further, medical texts and other general and specific medical information can be stored on the computer and made available to the expert analysis software for reference and for making a more complete and accurate diagnosis and treatment regimen.

In a preferred use, the system allows individuals to carry with them pertinent medical information and have that information available, and/or readily accessible to the medical practitioner. The smart card will have on it the personal information regarding the individual along with the information regarding the individual's last sixteen visits to a medical practitioner. When the individual visits a medical practitioner, the smart card is passed by a reader, the individual activates the biometric verification device so that the information on the smart card can be accessed, and the individual's information is read and can be displayed for viewing by the practitioner. The individual also can set a level of viewing for each practitioner. For example, the individual can allow a specific practitioner only to view the information, to read and manipulate the information on the computer, and to write new information to the smart card.

In a preferred embodiment, the practitioner can use the stored medical information to assist in diagnosing and treating the individual. Further, using the expert analysis module, the system can provide a separate and independent diagnosis and treatment regimen based on the individual's stored medical history and medical texts. The practitioner then can store the updated information back onto the smart card for later use.

In a preferred embodiment, the expert analysis module continually learns about the individual by updating old information with new information. In this manner, the expert analysis module allows for updated diagnoses and treatment regimens based on the latest information about the individual and, if used, the latest medical texts.

3. Specific System Features and Operation.

The present invention incorporates one or more data storage core sections on the smart card. In a specific embodiment for the purposes of this specification there are three data storage core sections on the smart card. These sections are:

1. The personal identification biometric authentication and other personal data sections are secured via biometric verification, such as fingerprint or voice recognition or retina scan, without the need for a mainframe or database of any kind. Up to forty-eight or more potential levels of security and encryption guarantee patient confidentiality. Secure files and data include the personal identification and Medical Passport and Alert section including chronic medication and contraindications, an Electronic Wallet that stores financial information such as banking details, or stored Value (for Electronic Funds Transfer for example), spouse's details, legal issues such as organ donor and living will, personal immunization records, and a confidential medical file (information that the smart card carrier does not want anyone to have access to without multiple security levels activated by the smart card carrier).

2. The medical benefit risk and management data section. These files are utilized to verify medical insurance details and benefits, options, capitated services, rules and regulations and details of the patient's primary care service provider, the last fifteen clinical encounter claim lines, medicine benefits and controls, benefit utilization records, cardholder specific template and security key data, etcetera. To facilitate better communication between healthcare professionals and administrative role-players, this section holds files for referrals, medical certificates and two message types for specific service site or specific doctor/patient. It further has the capability via an interface to generate a Chronic Medicine Application Form electronically at a provider site during a clinical encounter, without the need to go online or make a telephone call (EDI—Electronic Data Interchange). For further value at the employer-employee level, this section holds occupational health information, human resource (HR) management (worker's compensation) information, communicable disease information and other files and data. This data is only accessible to healthcare role players with the approval of the smart card carrier through a fingerprint biometric.

3. The e-purse section offers up to sixteen or more potential separate and secure e-purses per smart card. Use of these purses in conjunction with the comprehensive features described in sections 1 and 2 improves risk management by providing secure payment methods suited to a wide variety of benefits, options and other forms of funding types found in the healthcare sector. These include:
Token e-purse/s for use in closed environments; and
True e-purses based on interoperable standards agreed within the financial services sector.

The present invention can run on public or private (public or proprietary) smart card platforms. Such platforms comprise smart cards, readers, and other application development software, hardware, and communications technology. Following is an illustrative example of a suitable smart card platform.

Software:

a) A card data extraction software program to expedite the integration between the card and back-end practice management and EHR systems through the dynamic free issue software middleware link (SmartLink) software component of the invention.

b) An issuer card and software program to define the issuer and application keys.

c) An issuer initialization and personalization program that provides issuers with the capability to extract data, populate and personalize specific files directly from an existing database which is comprised of two software modules:
Software to load group data utilizing a data sample file from the issuer's database; and
Software to populate cards with data specific to a particular member, this includes the capturing of biometric data and other detailed personal data.

d) An issuer conversion software program to enable updates in respect of cards in use, without loss of captured data on a specific card.

e) An encryption/decryption program and interface to broadcasting software for the purpose of remotely writing data or deliver messages to specific cards at service provider sites, via Internet/intranet and/or exclusive satellite broadcasting and other switching systems.

f) A pharmacy extraction program that runs as middleware at the pharmacy level to interface with back-end software, read from, and write to the card.

These programs can be customized in order to meet the specific requirements of most issuers or other role players.

Readers:

a) The Saturn reader (Type A) is a fully integrated, highly secure smart card reader based on OTI's EYECON platform. The Saturn reader provides multi-function support in that it can support the ISO 14443 Type A, B and D modulation standards (including Mifare support) and ISO 7816 contact functionality. The Saturn reader incorporates flash memory with protected boot sector and programmable sector to enable secure online upgrades. OTI's matched antenna technology allows the distance between the antenna and the electronic circuitry in the reader to be as far as 33 meters (100 feet) apart.

b) Type B.I reader is a contactless reader that similarly incorporates flash memory to facilitate macro downloads.

Smart Cards:

a) Contactless only Type B or Type D cards or devices such as hand held tags, dog tags for the military, wrist bands, and key fobs; and

b) Dual interface (contact/contactless) Type B or Type D Multi-Cards.

The preferred microprocessor-based smart cards feature on-board operating systems, so they can easily incorporate new applications onto existing cards as well as revising existing applications in a secure manner via rapidly executable software routines. The ability to alter the functionality of the card through remote software upgrades enhances the flexibility of the card as an e-commerce or mobile computing platform and data repository. For example, up to sixteen different virtual cards with two hundred and fifty-six individual files can be run on any single smart card, providing for up to sixteen different applications (with sixteen different issuers).

Additional value-added applications can be accommodated on the same physical platform for the benefit of patients, service providers, employers and others business sectors. Issuers are potentially able to generate additional revenues by renting space on the card to third party issuers of additional applications.

The present invention, interfaced to the practice management component of the invention, or established and leading primary and secondary healthcare provider practice management software systems, provides users with a fully integrated and seamless solution. For example, on the pharmacy level the present invention can similarly be interfaced to leading pharmacy management software systems that can include electronic funds transfer (EFT).

The multitude of dumb cards (magnetic stripes) currently being issued to Medicare Rx beneficiaries and drug incentive plans is causing tremendous confusion to both the beneficiary as well as the pharmacist. There are too many different drug cards with different sponsors, benefits, discounts, and drug types. The patient-centric system of the present invention can consolidate all drug types, side effects, contraindications, discounts, and benefits on a single smart card that also stores usage records preventing duplicative and fraudulent use at point of supply. In addition and in combination with the expert system, data can be remotely input by the user/consumer at point of sale to provide the pharmaceutical industry with real-life information pertinent to patient-centric drug efficacy and side effects.

The present invention optimizes operational and treatment efficiencies and maximizes customer retention and loyalty. Coupled with a per-card/per-month flat fee pricing model, the present invention, properly deployed, has the ability to generate substantial return on investment for the issuer in a preferred business model.

4. General Example Embodiment of the System.

An illustrative embodiment of the present invention is to further the Health Information Technology (HIT) standard. Some of the most significant reasons for failure to adopt Health Information Technology (HIT) and resultant medical errors are financial, technical (lack of interoperable standards), and legal. The lack of interoperable systems and financial barriers are probably the most important reasons for the low percentage figures for implementation of HIT. The present invention is a cost effective solution for identification/biographic information, medical management, and e-purse applications. The present invention is based on extensive development to provide portability of personal electronic medical information (EMI) without the need for a central database. The present invention was developed to encompass, interact, and interface on primary, secondary and tertiary healthcare levels for physicians, hospitals, pharmacies, and all ancillary providers. Effective communication is encouraged and established between all role-players to improve population health in the community network.

The identity and authentication of a patient and his/her healthcare benefits are recognized by the present invention at the point of service. Secure effective data is captured at the time of the clinical encounter, and based on standardized international codes for diagnosis, procedures, treatments, and medications. Medical, clinical, and administrative management systems can be linked to the system. The method secures effective database storage, management, and data mining. Clinical guidelines, protocols, and patient doctor performance based on international standards, statistics, and outcome reviews are incorporated through an expert system. The best clinical and financial risk management tools and HIT standards are available. The system facilitates fast effective payment in a variety of models for services rendered at the service provider level, based on various benefit options, protocol compliance, and performance of patient and service provider.

Electronic access of patient information facilitates updating of records simply through the presentation of the card to the reader, and functionality that features interface to online eligibility and service provision parameters. This data is compared and validated with the patient's status embedded on the card. At the clinical encounter level the card can be configured to act as the initiator of a clinical encounter, ensuring that the attending physician/pharmacist or provider is informed as to contraindications, chronic medication, and allergies. New clinical encounter data is written to the card utilizing standard international codes to indicate the date, patient-number, service provider-number, codes for diagnosis (CPT4, ICD9/10), clinical services, procedures, medications (e-scripting) and cost. This enables the cardholder to carry his/her critical and important identification, clinical, and other data on a single contactless card for use within the medical community without the need for duplicative data capture/re-entry. Access to such data is protected through the use of various security features, such as fingerprint verification, voice recognition and/or retinal scans. The need to go online, or access a database is in most cases eliminated. All available options, such as managing fee for service, group and individual capitation, short term insurance, hospital plans, multiple insurance plans, and combinations of the above are customizable.

The present invention allows not only for secure portability of basic information on the card but will also connect to an individual data repository (databank) in which the patient's electronic medical record (EMR) is stored. Access to the individual's EMR's is authorized by the individual at point of service through presentation of the card and identity verification via fingerprint and/or voice biometric. The patient decides the level of access he/she is prepared to allow, i.e. view only, view and update, full access, etcetera. In today's world of computer hackers managing to access supposedly secure databases, even at the US Pentagon level, the present invention makes it totally impractical and cost prohibitive for hackers to hack into millions of individual repositories versus breaking into one single database of information about millions of people.

In a recent study Emory University reported on their website of Health Affairs that the cost of health care is an increasingly critical issue for families, employers, and policy makers. Health care is projected to account for 15% of the US gross domestic product in 2004, up from 11% in 1987. The cost of health insurance during the past three years has jumped by an average of 12.5% each year. The present invention incorporates the use of an expert system (reasoning) in which generic clinical guidelines and protocols are embedded. On the one hand this assists the healthcare provider to make consistent decisions and, on the other hand the system, through its intelligent learning machine, dynamically evolves the generic base into a patient-centric knowledge base that tracks patient specific reaction to treatment and progress. In essence the system automatically steps through a symptom/derivation/diagnosis/treatment map. Linking this reasoning map, not only to the patient's personal EMR repository (visibility), but also to the patient's personal smart card (portability) that provides care providers, other than the patient's personal care provider, access to more than just a clinical diagnostic code but provides the healthcare professional the previous provider's logic in determining diagnosis and treatment (visibility).

Doctors will eventually benefit from EMR, but the big picture requires massive implementation, and the savings that can be derived from group participation. In fact just the documentation of eligibility, co-pay, capitation, etcetera before medical service is provided in out-patients and inpatients facilities requires the doctor's staff to frequently verify the patient status by phone, fax, or paper, and collect prior medical history before the service. This is a tedious and frustrating affair requiring personnel and extra administrative and personnel costs.

Collection of regulated medical fees, lab fees, and other procedural fees is a daunting task, which could be handled electronically more efficiently. In fact insurers have lowered the fees reimbursed to the doctor for operating an efficient electronic medical practice by decreasing the service fees paid by bundling independent services without any consideration for the costs involved, or the liability for medical errors that may result in the daily physician and staff stress of caring for patients. A 2004 study by the Center for Information Technology Leadership cited 2.1 million patients suffered from medical errors due to drug reactions when EMR could have warned about drug interactions if HIT technology had been employed.

The present invention includes the entire picture of the health care community, and interfaces with hospitals, ancillary providers, retail pharmacies, telemedicine, and even schools (for the documentation of immunizations). The US federal government has launched incentives to implement widespread adoption of electronic health records and a new nationwide interoperable health information network, especially for rural areas. The smart card of the present invention allows seamless integration with existing practice management systems in medicine and provides the patient with a medical passport that he/she can carry and utilize like a credit card, but with more security in that his/her identity is established with a fingerprint biometric, and in the case of a child, or impaired individual, parent or guardian, two fingerprints may be required, a personal identification number (PIN), or password.

The present invention can improve the quality of care by giving the patients and their doctors or other care providers a complete, up-to-date view of a patient's medical history, admissions, procedures, medications, immunizations, allergies, labs i.e. blood type for emergencies, and recently even DNA profile for special hazardous circumstances in the military or industry. Patients control and manage these records from different providers that include their personal records, which can be updated as needed and with each clinical encounter. The present invention can be used as an e-purse with credits or debits that may include up to fifteen other dumb cards (magnetic stripe). The new prescription card programs can be utilized for discounts, Medicare, insurance, etcetera, and even transmission of the actual prescription on the card to a pharmacy of choice after the doctor visit with accounting functions, such as decrementing/incrementing benefit balances, being performed and recorded on the card via the on-board microprocessor and operating system. In this way the patient does not have to carry many different dumb cards for each insurer or application. With the fingerprint, even a driver's license and photograph could be added to the smart card to increase administrative efficiency and establish identity. The innovation could even be added to a passport, or a passport be embedded on the card. Many other similar services for medical records are available on the Internet, but frequently computer access for the consumer is a problem and implementation is eventually paper based.

The present invention also allows the health care professional and his or her staff to spend more time with the patient. The unique aspect of the smart card is that it can operate independently or with existing systems or databases. The card is updated with each physician, hospital admission, pharmacy visit, etcetera. The same microprocessor can hold up to fifteen or more recent visits to the provider that is important for referrals, return to work statements (limitations), or lab procedures. A valued module that connects to the card is the expert system, a patient-centric, case based data mining process which learns with each patient encounter and cross references the data by disease, outcome, therapy (drug/prescription), or procedure etcetera. This module is based on clinical protocols that can be extremely helpful in monitoring trends and risk management. The expert system could solve many of Medicaid's case/disease management problems, i.e. the top five most expensive medical conditions reported in the Emory study were (I) heart disease, (2) trauma, (3) cancer, (4) pulmonary conditions and (5) mental disorders. The e-purse provision of the present invention could settle payment to the providers, vendors, hospital, clinics and others electronically without expensive third party administrators, and avoid lost, erroneous, fraudulent claims, requiring delayed paper checks that are sent by snail mail.

5. Specific Example Embodiment of the System in Connection with the Pediatric Psychiatry Field.

An example of an innovative business model for the present invention is in the pediatric psychiatry field. This model involves the community, the Internet, the hospital, pediatricians, clinical psychologists with master degrees, and clinical psychologists with doctorate degrees. Even the schools have access to triage mentally ill children and learning disabilities for diagnosis and treatment with referral to these professionals. Objective follow-up, physical assessments, psychotherapy of the child with parent or guardian interaction, are accomplished. Progress reports on drug therapy, teacher monitoring, and psychological testing are resulting with improved outcomes.

The expert system used in the present invention can be informally described as a process for collecting relevant information and objectively, systematically and consistently using this information to monitor and maintain a patient's health. By doing so, the creation of a historical database will allow the creation of a better predictive health maintenance schedule for the patient.

The method and system can be in many different forms, a basic version of which comprises the steps of:

(1) Obtaining past and current medical information from the patient;

(2) Entering the information or updating the appropriate fields for downstream predictive decision or modeling of the patient's health;

(3) Applying to the data a series of database algorithms, probability matrices, and solutions to determine an immediate situational response and activity directives for diagnosing and treating the patient;

(4) Retrieving real time, or near real time, updates of treatments regarding the patient;

(5) Retrieving past physicians' and other health practitioners' comments regarding the patient;

(6) Receiving current comments regarding the patient; and

(7) Allowing updates to the database as pertinent to the maintenance and monitoring of the patient's current health.

An additional feature of the invention allows the retrieval of all information for data mining and cost benefit modeling.

The first step can include obtaining health and treatment information from the patient, including a current medical checkup.

The second step, after the current patient medical information has been received, can include entering or updating the appropriate fields for downstream predictive diagnosis modeling pertinent to the particular patient.

The third step can include the application of a series of database algorithms, probability matrices, and database solutions to the data collected to determine the immediate situation responses directives. At this step, the invention can provide a diagnosis of the patient's condition based on past diagnoses and treatment, as gleaned from the inputted data regarding the patient's past medical examinations and treatments. In one embodiment, the system can learn from previous applications. As an example, in this step, the invention can alert the medical practitioner that the patient has a history of high blood pressure, that the patient's blood pressure is elevated based on the current medical examination, and that the patient responds to a certain type of treatment or drug based on the database of information of past treatments and diagnoses for this patient contained on the smart card. Likewise, the invention can alert the medical practitioner that the patient's symptoms may relate to another possible illness.

The fourth step can include inputting and receiving real, or near real time, updates of treatments given or recommended. For example, recent surgeries, prescriptions, or other medical treatments performed on the patient can be inputted. Such inputs can be either per examination or per time period.

The fifth step can include receiving comments from the expert system of the invention on the treatments and diagnoses recorded by the invention. For example, if a certain preventative treatment (annual Pap smear, mole examination for cancerous growth, dental checkup) is required on a patient, the invention can notify that such a treatment is needed. The invention at this point can also generate scheduled preventative or necessary treatments.

The sixth step can include receiving comments regarding the patient from the patient, treating physicians, and people with first-hand knowledge of the patient.

The seventh step can allow updates to the database as pertinent to the treatment or diagnosis in question. The database is updated based on probable future treatments and diagnoses, or preventive treatment recommendations, for the patient. Thus, based on the pattern of previous diagnoses and treatments of the patient, the database will contain additional information as the method is used.

An eighth additional step can include allowing a user to retrieve past information and to examine trends in the health and treatment of the patient. For example, the data collected for prognostic treatment decisions can be mined to develop condition based models to determine the physically, mentally, emotionally, and/or economically most viable treatment option as it pertains to the patient, i.e., a determination of preventative (to avoid a medical condition) versus actual (to treat an actual condition) treatment based on current health maintenance.

The process uses a number of available agents as components of the whole. One agent is a statistical modeling technique for the prediction of events. A commercial example of this agent is EXAKT®, which is incorporated herein by this reference, which is used for machinery but can be adapted for use with humans. A second agent is an adaptive expert system shell that has the potential of widening the electronic communication link between the user and the customer. A commercial example of this agent is SOLVATIO®, which is incorporated herein by this reference.

Somewhat more specifically, input data can include parameters such as height, weight, blood type, cholesterol levels, blood pressure, heart rate, and the like, including but not limited to past treatments and diagnoses. Once this data is inputted, the patient's current medical condition then can be compared to this base data and a determination made as to whether the patient is still at the baseline or if some aspect of the patient's health has changed.

The information thus gleaned is sent or inputted into a computer management system (CMS). The CMS module also can receive direct input from medical practitioners and from the expert system statistical data module. The CMS module then can be used to construct a health maintenance schedule for the patient.

Information regarding the health maintenance schedule from the CMS module is sent or inputted to a statistical modeling technique module for the prediction of medical conditions, such as the EXAKT® agent disclosed above, and to an adaptive expert system shell that has the potential of widening the electronic communication link, such as the SOLVATIO® agent disclosed above.

The statistical modeling technique module and the adaptive expert system shell analyze various aspects of the data from the CMS module, such as treatment and diagnoses information, past drug prescriptions and surgeries, and the like, and provide a preliminary diagnosis of the patient. For example, by combining suggested preventative maintenance activities (that is, suggested preventative health measures such as limiting salt intake, taking a vitamin or aspirin each day, exercise) and historical medical data (that is, when and what treatments have been performed on the patient), the system develops a health maintenance schedule for the patient. This health maintenance schedule may be the same as or different from the maintenance schedule suggested by the physician, or the historical health maintenance schedule, and is based on the actual factors pertaining to the particular patient, and not to a generalized group of like patients.

The analysis and scheduling can increase physician productivity, as the health maintenance schedule will be more exact and more relevant to the individual patient. The system can predict both health maintenance that needs to be performed and potential health problems that may arise based on a historical and real time snapshot of the particular patient.

A web-enabled HTML viewer (a GUI—graphical user interface) allows the user to interact with the system. Through the GUI, the user can review any number of data, such as the data inputted into the system, the scheduled health maintenance, the historical health maintenance, and/or the health maintenance schedule developed by the system. Further, the system provides a result condition prognostic for the patient, which helps the user optimize the operation and maintenance of the patient. Through this result condition prognostic, the user can decide what, if any, health maintenance actions to take.

Further, a what if module can be used to set up various different scenarios. The user can use the what if module to obtain an indication of whether the patient may need earlier or later treatments based on certain health maintenance assumptions. For example, if the system indicates that a certain health maintenance activity (an electrocardiogram or an angioplasty) should be carried out every ten years, the user can use the what if module to obtain an indication of whether waiting for twelve years would adversely affect the patient.

The entire process is software driven, and thus is efficient and rapid. Further, the statistical modeling technique module for the prediction of treatment and the adaptive expert system shell are self-learning, and provide the system with the ability to revise the health maintenance scheduling in real time for the particular patient. As such, the health maintenance scheduling is not set for a patient, but can change as the patient changes over his or her lifetime.

As can be seen, the system drills down to review the data from a particular patient, and not just a general patient (the textbook patient). For example, the system reviews the particular patient and develops a health maintenance schedule for that particular patient, rather than averaging data for all patients contained in the system. This allows greater efficiency and optimization for the operation and health maintenance for each individual patient.

The invention also comprises a business method of implementing the process. Such a business method can allow a separate company or a user to monitor and maintain the units. For a separate company, this would allow for an income stream for providing the service. For the user, this would allow savings due to more efficient and economical monitoring and maintenance.

6. Specific Example Embodiment of the System in Connection with the Drug Treatment and Delivery Field.

Following is an example of an innovative business model for the present invention is in the drug treatment and delivery field.

Data is powerful in the medical industry. However, collecting, interpreting and administering data is cumbersome and time consuming for the provider, and most other role players. Adding to this dilemma, conflicting objective and subjective expert opinion, combined with fragmented and disparate data collection and evaluation technology, collectively contribute to incorrect, or at best contentious decision-making in the proverbial “trenches” of medicine, even when data is recorded. Harnessing, then harvesting knowledge accurately gathered via interactive dialog into a central knowledge pool, is the answer that provides the base from which positive, issue-centric, and validatable decisions could be made in a dynamic “real-world” environment.

Within the healthcare arena, there appears to be a tremendous lack of coordinated collaboration between the different role players. This lack of centralized structure unfortunately leads to errors resulting in serious consequences; consequences that may have been avoided through a process of “cradle-to-grave” measurement, monitoring and management. In other words, we fail to learn from our errors until it is too late for definitive action, or a resultant death occurs. Case in point is the apparent lack of ongoing real-world data gathering and assessment of effectiveness and negative reactions to drugs on a patient-centric basis. Whilst effectiveness and negative reactions are recorded during the investigative process, recordal of real-world, real-time patient-centric data is not gathered subsequent to drug approval until severe complications are observed. Marketing errors are usually followed by class action liability lawsuits which threaten the entire investigative (drug) process and financial health of the industry. Some authors feel that the entire process is aggravated by the controversial direct-to-consumer advertising of drugs. This marketing strategy after approval by the FDA, may affect or influence the selection of the patient population prescribed the drug in which complications develop.

The key to improving outcomes in the healthcare continuum lies in adopting an expert system platform that embodies a dynamic “learning machine”—a virtual adaptive “engine”. More importantly such technology should have the ability to embody scientific formulas and rules, using complex mathematical algorithms, whilst providing a user-friendly interactive front-end for conversation-style dialog, and “feeding” an administrative process. This would establish a central data-gathering and interpretation platform, drug-specific, that dynamically evolves during the trial phase, and adapts by intelligent learning of case-based outcomes, throughout the life of the drug. By developing multi-dimensional “boundary tables” (high/low thresholds), variables and weighted covariates using mathematical computation as condition markers, a scientific base is established to discern patient reaction, whether adverse or positive. Professional feedback from the prescribing physician (through an interactive web-based checklist) before formally scripting is supported by patient input/response (via the internet or at the time of receiving the prescribed drug/s). The original trial-phase “knowledge base” will keep growing and adapting to the dynamics of the environment and note patient compliance, or otherwise. Even in drug investigative failures, data could be mined in disease combinations, for genomics, for other applications, or options for designer drug implications i.e. affect on BP, blood sugar, etc. to broaden the claim(s).

The challenge, simply stated, is to provide all role players (researchers, scientists, physicians, pharmacies, investigators, and the patient) with a single-source “engine” configurable to suit the requisite level of situational (professional and user) complexity/simplicity via an easy-to-use interactive interface.

As a first phase response, and proof of concept, to this challenge, J4 MediSmart has developed, and is in the final stages of deploying, a system catering to Behavioral Medicine to address the above issues. The initial approach in this discipline involves three tiers of knowledge. The first tier is the Pediatric Psychiatrist, and the second tier in this instance consists of clinical psychologists, masters and PhD's of psychology. The third tier involves community resources, more specifically, social workers, case workers, school counselors and primary care providers (family practitioners, internists, pediatricians, etc.) human resources to “feed” into the knowledge base. In tandem, this data combine to provide extremely powerful “real-life”, near real-time patient-centric drug/treatment efficacy data, as well as an immediate response for dosage adjustment and/or treatment modification when required by the attending certified specialist. All three tiers of professionalism “feed” the knowledge base. It is hoped that this bottom-up approach will provide the pharmaceutical industry with real-world visibility of the potential usefulness and power of the J4 end-to-end solution. The system, in deployment for behavioral drugs, combines:
An adaptive expert system comprising of:
   A rules-base component
   A case-based component
   A progress evaluation component i.e. collects patient vitals, markers, etc., and provides a suggested action list at time of encounter.
   Access for “community resources” to evaluate/re-evaluate patient condition severity (progression/regression) through a simple to use 0-1 0 condition severity matrix.
   A “learning machine” to adapt to patient-centric reaction data and environmental, situational, and condition dynamics.
A Smart Card that:
   Positively identifies the patient, both at clinical encounter level, as well as at the dispensing pharmacy/entity
   Stores known allergies, contra-indications, and medical history, visible to both the prescribing doctor as well as the dispensing pharmacist.
   Facilitates multiple applications, users, and drug/financial companies on the same card—all totally secure from one another.
   Stores multiple clinical encounter information by diagnostic code (ICD9/DSM-4, etc.), and facilitates the link (via a .dll) to patient EMR's on back-end practice management systems.

Case-based, patient-centric data thus acquired in practice, can be seamlessly interfaced with source benchmarks (boundary tables) at pharmaceutical company level to determine global (not in geographic sense) statistical outcomes, but more importantly can generate automated dosage adjustment suggestions based on factual referencing. Of obvious value to the “serious” physician are interpreted data to secure positive outcomes, error elimination/reduction, risk management, and the experience of managing minor/major drug side effects. Of obvious value to the pharmaceutical company is this data to prompt timely intervention to adverse reactions when indicated and develop a central electronic source for the recording of ongoing drug efficacy verification and validation of data, data that can not be deleted or changed.

Once validatable data is available we combine a statistical modeling tool to correlate past events and past observations (encounters, measurements, etc) in a statistical model. That model is then used (preferably automatically as an “intelligent agent”) for predictive decision support each time a new set of measurements becomes available in the central database. To build the model requires good and valid historical data based on literature, previous confidential drug studies, and/or other sources. MediSmart's knowledge comes directly from such harnessed data that in the past was lost or difficult to retrieve.

In the real world good historical event data is not always available. Generally, many failure modes (aka diagnoses, results, advisories, etc) can be recognized and tracked by using human expert reasoning, others require a sophisticated statistical approach (e.g. proportional hazard modeling), while still others can be detected using the statistical approach of case based reasoning (expert system). Some of the most effective applications of artificial intelligence are in fact hybrid systems of two or more AI components where the output of one feeds the input of another. The MediSmart expert system, as a spin-off benefit of its case based reasoning process can collect great quantities of event data which are in an ideal format for the development of proportional hazard models to predict negative reactions within a statistical probability range.

In essence, the e-MediSmart process provides all role players (pharmaceutical company, provider, pharmacy, and patient) with technology that “funnels” all pertinent data to a central, cohesive, intelligent, and dynamic adaptive platform for: data storage, evaluation, monitoring, interpretation, and proactive action (if/when required) throughout the entire lifecycle of a drug—from scientific research to clinical trial to limited controlled use to mass consumption.

TABLE 1
The MediSmart process:

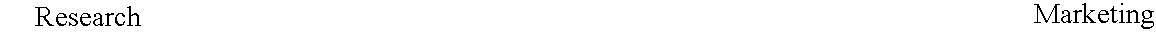

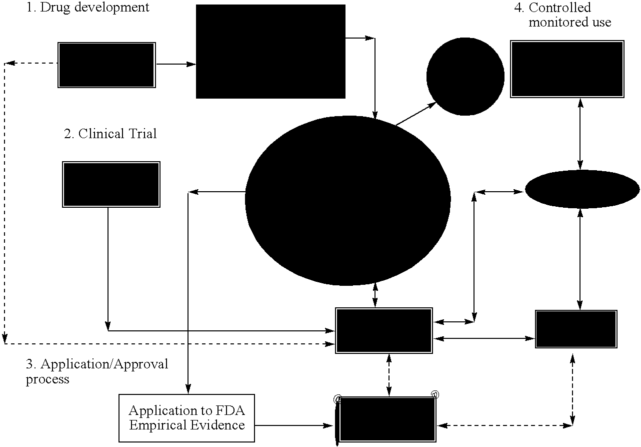

TABLE 2
The J4 MediSmart method

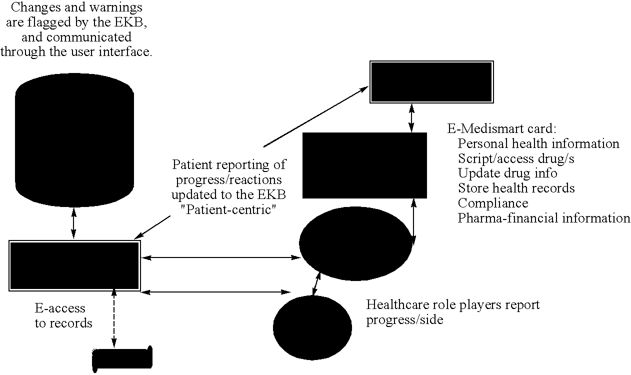

Measurements, Monitoring, and Action:
Occurrence of labeled side effects; patient-centric.
Occurrence of off-label/new side effects; patient-centric
Immediate remote notification of labeling, cancellation of drug dispensing, when negative reactions are indicated through mass online notification to pharmacies, physicians and consumers.
Remote “locking-out” of drug delivery through broadcasting to patient card.
Patient compliance.
Physician measurement of the effectiveness of the drug—reduction of disease level/s on a patient-centric and group profile basis.
Accuracy of conceptual design and formulation.
Compliance with FDA rules and regulations throughout the lifecycle of the drug.

The Technology: Dynamic Expert System Components (DESC)
1. Adaptive Expert System—MedSolve
2. Statistical Modeling System—MedExakt
3. Smart Card—eMediSmart Card

[image]

MedExakt: Statistical Probability Modeling.

Provides statistically objective risk-level decision support for more consistent and effective treatment and outcome control, using Proportional Hazards Modeling (Weibull)

### Example of PHM Hazard Function

β=3, η=30,000, γ₁=0.15 (e.g. iron parameter), γ₂=0.5 (e.g. marker parameter=lead)

$$h ( t \vert {Z}_{1} ( t ) , {Z}_{2} ( t ) ) = ( \frac{3}{\text{30,000}} ) {( \frac{t}{\text{30,000}} )}^{3 - 1} {ⅇ}^{0.15 {Z}_{1} ( t ) + 0.5 {Z}_{2} ( t )}$$

Let Δt=100. Some examples of hazard:

| t | Z₁(t) | Z₂(t) | Hazard at t × 100 |
|---|---|---|---|
| 1000 | 10 | 2 | 0.000135 |
| 1000 | 15 | 4 | 0.000779 |
| 12000 | 10 | 2 | 0.0195 |
| 12000 | 15 | 4 | 0.122 |

E.g., at t = 12000 h, and Z₁ = 15, Z₂ = 4, hazard is 0.00122, and Probability of failure in 100 h is 0.00122 × 100 = 0.122.
If β = 1, hazard does not depend on time, only on measurements

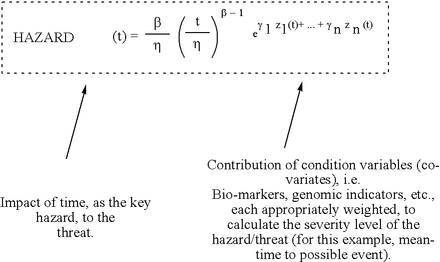

[image]

[image]

[image]

The above detailed description of the preferred embodiments is for illustrative purposes only and is not intended to limit the scope and spirit of the invention, and its equivalents, as defined by the appended claims. One skilled in the art will recognize that many variations can be made to the invention disclosed in this specification without departing from the scope and spirit of the invention.

What is claimed is:

1. A computerized system for monitoring and maintaining the health of a patient comprising the steps of:
a. obtaining parameter data from the patient and inputting the parameter data into a computer database;
b. analyzing the parameter data using a computerized statistical modeling technique module and a computerized adaptive expert system shell for the prediction of a health event in the lifetime of the patient; and
c. using the analyzed data to develop a health maintenance schedule for the patient.

2. A computerized system for monitoring and maintaining the health of a patient comprising the steps of:
a. obtaining parameter data from the patient pertaining to the status of at least one health factor of the patient and inputting the parameter data into a computer database;
b. analyzing the parameter data using a computerized statistical modeling technique module to develop a health maintenance schedule for the patient;
c. analyzing the parameter data using a computerized adaptive expert system shell for the prediction of a health event in the lifetime of the patient; and
d. providing the analyzed data to a user to implement a health maintenance and monitoring schedule for the patient.

3. A computerized system for monitoring and maintaining the health of a patient comprising the steps of:
a. obtaining parameter data from the unit and inputting the parameter data into a computer database;
b. analyzing the parameter data using a computerized statistical modeling technique module and a computerized adaptive expert system shell for the prediction of a health event in the lifetime of the patient;
c. using the analyzed data to developed a first health maintenance schedule for the patient; and
d. providing the analyzed data to a user to implement a second health maintenance and monitoring schedule for the patient.

4. A computerized system for monitoring and maintaining the health of a patient comprising the steps of:
a. obtaining parameter data from the unit and inputting the parameter data into a computer database;
b. analyzing the parameter data using a computerized statistical modeling technique module and a computerized adaptive expert system shell for the prediction of a health event in the lifetime of the patient;
c. using the analyzed data to develop a first health maintenance schedule for the patient;
d. providing the analyzed data to a user to implement a second health maintenance and monitoring schedule for the patient; and
e. allowing the user to alter the parameter data to create an alternate hypothetical health maintenance and monitoring schedule for the patient.

5. A computerized system for monitoring and maintaining the health of a patient comprising:
a. a first means comprising a smart card having a microchip with memory for storing parameter data about the patient;
b. a second means comprising a smart card reader for receiving the parameter data about the patient from the first means; and
c. a third means comprising a computer for analyzing the parameter data using a computerized statistical modeling technique module and a computerized adaptive expert system shell for the prediction of a health event in the lifetime of the patient,
wherein the analyzed data is used to develop a health maintenance schedule for the patient and to allow a user to implement a health maintenance and monitoring schedule for the patient.
